(19) United States
(12) Patent Application Publication
(10) Pub. No.: US 2026/0224288 A1
(43) Pub. Date: Aug. 6, 2026

(54) SYSTEM AND METHOD FOR AUGMENTED REALITY MEDICAL REGISTRATION WITH ADAPTIVE FIDUCIAL TRACKING

(71) Applicant: MEDIVIEW XR, INC., Cleveland, OH (US)

(72) Inventors: Jeffrey Harold Yanof, Solon, OH (US); Peter Nicholas Braido, Wyoming, MN (US); Seyedsina Shirinpour, Minneapolis, MN (US); Ross Daniel Hinrichsen, Plymouth, MN (US); Cameron Nicholas Mar, Cleveland Heights, OH (US); Philip Lyman Deming, III, Somerset, CA (US); Gregory Pak Lung Wong, Folsom, CA (US)

(21) Appl. No.: 19/532,247

(22) Filed: Feb. 6, 2026

Related U.S. Application Data
(60) Provisional application No. 63/754,785, filed on Feb. 6, 2025.

(51) Int. Cl. A61B 34/10; A61B 34/20; A61B 90/00

(52) U.S. Cl. CPC A61B 34/10; A61B 34/20; A61B 90/361; A61B 2034/105; A61B 2034/2051; A61B 2034/2055; A61B 2090/365

(57) ABSTRACT

A system for augmented reality visualization during medical procedures includes an augmented reality display system that displays holographic representations of internal anatomical structures overlaid in spatial alignment with physical anatomy of a patient. A registration device positioned on the patient includes a tracking component that generates tracking data indicating position and orientation of the registration device. An imaging system acquires medical imaging data of the patient's internal anatomical structures. A computer system receives the medical imaging data and tracking data, determines spatial alignment between the medical imaging data and the patient based on the tracking data and a known position of the registration device, computes registration transformations between an imaging coordinate system and a physical coordinate system of the patient, and transforms the medical imaging data to enable display of holographic representations in correct spatial alignment with the patient's physical anatomy.

### CROSS-REFERENCE TO RELATED APPLICATIONS

This application claims the benefit of U.S. Provisional Application No. 63/754,785, filed on February 6. 2025. The entire disclosure of the above application is incorporated herein by reference.

### F IELD

The present technology relates to extended reality systems for medical applications, and, more particularly, to registration systems for aligning virtual imaging data with physical patient anatomy during medical procedures.

### INTRODUCTION

This section provides background information related to the present disclosure which is not necessarily prior art.

Extended reality systems for medical procedures enable surgeons and interventional radiologists to visualize internal anatomical structures by projecting holographic representations that align with the body of the patient. Achieving this alignment requires establishing correspondence between multiple coordinate systems, for example, the tracking system monitoring instruments, the display system presenting holograms to operators, and the imaging coordinate system containing diagnostic scan data.

Registration processes can create substantial workflow burdens. Operators must prepare patients for imaging by placing reference markers at specific anatomical locations, coordinate imaging acquisitions to capture these references, and execute multi-step registration procedures that verify spatial correspondence between physical and virtual spaces. Each step introduces potential delays, particularly when imaging occurs days before procedures and markers must be removed and repositioned based on fading skin marks or anatomical estimates.

Tracking system port limitations constrain procedural capabilities. Medical navigation platforms provide limited connections for sensor inputs, forcing allocation choices between registration infrastructure and procedural instruments. When complex procedures require tracking ultrasound probes, ablation devices, biopsy needles, and vascular catheters simultaneously, port scarcity creates operational bottlenecks that limit procedural options or force sequential rather than concurrent device tracking.

Component costs accumulate across patient volumes. Registration infrastructure combines precision sensors, visual targets, specialized housings, sterile packaging, and associated hardware into systems that serve single procedures. Manufacturing demands, quality control requirements, and regulatory compliance contribute to per-procedure expenses that affect healthcare economics and patient access to advanced visualization technologies.

Geometric constraints limit procedural flexibility. Registration approaches require positioning reference components according to predetermined spatial patterns to ensure adequate distribution for computing accurate transformations. These spatial requirements may conflict with anatomical contours, imaging field-of-view boundaries, or surgical access needs. Cone beam computed tomography systems maintain relatively constrained fields of view compared to multi-detector systems, creating tension between reference marker spacing requirements and imaging coverage capabilities.

Registration workflows interrupt procedural flow. Operators position components, verify system connectivity, confirm geometric relationships meet tolerance requirements, and validate spatial correspondence before proceeding. Discrepancies in placement, target recognition, or sensor readings trigger troubleshooting sequences that extend procedure time and increase cognitive burden during time-sensitive interventions.

Pre-procedural preparation demands intensify workflow complexity. Imaging technicians must place markers at precise locations before scanning, document positions, and ensure visibility throughout acquisition. Imaging data undergoes segmentation processes that identify and label anatomical structures, markers, and pathological features. Segmentation often requires external services or specialized software, introducing delays between imaging and procedural readiness that impact scheduling flexibility and treatment timing.

Patient movement during procedures challenges registration stability. Respiratory motion, table adjustments, and natural patient shifting affect spatial relationships between external reference markers and internal anatomy. Systems relying on skin-mounted references assume skin position correlates with organ position, but respiratory excursion, tissue deformation, and patient repositioning disrupt this assumption. Maintaining accuracy throughout dynamic procedures requires either frequent re-registration or sophisticated motion compensation, both adding operational complexity.

Field-of-view limitations constrain imaging compatibility. Different imaging modalities provide varying coverage volumes, with intraoperative cone beam systems offering smaller fields than pre-operative multi-detector scanners. Registration systems designed for larger field coverage may exceed intraoperative imaging capabilities, forcing compromises between spatial distribution requirements and imaging constraints. These limitations become particularly acute when transitioning between imaging modalities during procedures.

Ergonomic considerations affect operator performance. Managing hardware components, routing cables, verifying system states, and monitoring multiple sensors all demand attention that could focus on primary procedural objectives. Physical presence of registration infrastructure on patient anatomy creates obstacles for instrument manipulation, probe positioning, and sterile field maintenance. Cable management requires careful planning to avoid interference with operator movements, camera sightlines, and patient positioning.

Accordingly, there is a continuing need for registration systems that maintain accurate spatial alignment while reducing setup complexity, decreasing component costs, accommodating diverse imaging modality constraints, minimizing pre-procedural preparation requirements, and enhancing procedural ergonomics and workflow efficiency.

### SUMMARY

In concordance with the instant disclosure, registration systems that maintain accurate spatial alignment while reducing setup complexity, decreasing component costs, accommodating diverse imaging modality constraints, minimizing pre-procedural preparation requirements, and enhancing procedural ergonomics and workflow efficiency have surprisingly been discovered.

The present technology includes articles of manufacture, systems, and processes that relate to registration devices integrating various tracking capabilities for establishing spatial correspondence between multiple coordinate systems in extended reality medical guidance applications.

In one embodiment, a system for augmented reality visualization during medical procedures on a patient can comprise an augmented reality display system that can display holographic representations of internal anatomical structures of the patient overlaid in spatial alignment with physical anatomy of the patient. The system can include a registration device that can be positioned on the patient, where the registration device can include a tracking component that can generate tracking data indicating position and orientation of the registration device relative to the patient. An imaging system can acquire medical imaging data of internal anatomical structures of the patient. A computer system can receive the medical imaging data from the imaging system, receive the tracking data from the registration device, determine spatial alignment between the medical imaging data and the patient based on the tracking data and a known position of the registration device on the patient, compute registration transformations between an imaging coordinate system and a physical coordinate system of the patient, and transform the medical imaging data using the registration transformations to enable the augmented reality display system to display the holographic representations in correct spatial alignment with the physical anatomy of the patient during medical procedures.

In another embodiment, a method for medical registration can comprise positioning a registration device on a patient, where the registration device can comprise tracking components. The method can include determining a position of the registration device relative to the patient based on tracking data from the tracking components, and registering medical imaging data to the patient based on the determined position of the registration device. The method can further comprise determining position and orientation of a medical instrument based on tracking data from a probe bracket attached to the medical instrument, and displaying, by an augmented reality system, augmented representations based on the registered medical imaging data and the position and orientation of the medical instrument.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic view of a system for augmented reality visualization during medical procedures according to one embodiment of the present disclosure.

FIG. 2 is a perspective view of a registration device positioned on a patient.

FIG. 3 is a perspective view of the registration device of FIG. 2 further depicting a holographic representation of an ultrasound image aligned with anatomy of the patient.

FIG. 4 is a perspective view of a registration device including an electromagnetic sensor and multiple optical markers.

FIG. 5 is a perspective view of another registration device including an electromagnetic sensor and a single optical marker.

FIG. 6 is a cross-sectional view of the registration device of FIG. 5 depicting the electromagnetic sensor.

FIG. 7 is a perspective view of an ultrasound probe mounted in a probe bracket.

FIG. 8 is a perspective view of a registration device including optical markers and radiopaque markers.

FIG. 9 is a flowchart illustrating a method for medical registration according to one embodiment of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1–10, or 2–9, or 3–8, it is also envisioned that Parameter X may have other ranges of values including 1–9, 1–8, 1–3, 1–2, 2–10, 2–8, 2–3, 3–10, 3–9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures.

Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

As used herein, the terms “interventional device” or “tracked instrument” refers to a medical instrument used during a medical procedure.

As used herein, the term 'tracking system' refers to something used to observe one or more objects undergoing motion and supply a timely ordered sequence of tracking data (e.g., location data, orientation data, or the like) in a tracking coordinate system for further processing. As an example, the tracking system can include electromagnetic tracking that can observe an interventional device equipped with a tracker, optical tracking that can detect reflective markers or light sources, fiber optic shape sensing for flexible catheters and instruments within the body, electro-anatomical mapping, impedance-based tracking, lidar or other tracking modalities that can observe the robotic or manual arm system as it moves or is positioned or interventional device as it enters, moves through and/or in, and exits a patient's body, as well as while outside of the patient's body.

As used herein, the term "tracking data" refers to information recorded by the tracking system related to an observation of one or more objects undergoing motion, such as patient tissue, registration devices, medical instruments, interventional devices, or combinations thereof.

As used herein, the term “head-mounted device” or “headset” or “HMD” refers to a display device, configured to be worn on the head, that has one or more display optics (including lenses) in front of one or more eyes. These terms may be referred to even more generally by the term “augmented reality system,” although it should be appreciated that the term “augmented reality system” is not limited to display devices configured to be worn on the head. In some instances, the head-mounted device can also include a non-transitory memory and a processing unit. An example of a suitable head-mounted device is a Microsoft HoloLens® head-mounted device (Microsoft, Redmond, Washington).

The extended reality system can enable visualization through various modalities including but not limited to: augmented reality (AR) where virtual content is overlaid on the physical world, virtual reality (VR) where camera passthrough enables viewing of the physical environment within an immersive virtual space, and mixed reality (MR) where physical and virtual elements are combined in an interactive environment. The system can support multiple display types including head-mounted displays, handheld devices, and fixed position see-through displays while maintaining registration between virtual content and physical objects.

As used herein, the terms “imaging system,” “image acquisition apparatus,” “image acquisition system” or the like refer to technology that creates a visual representation of the interior of a patient’s body, or a portion thereof. For example, the imaging system can be a computed tomography (CT) system, a fluoroscopy system, positron emission computed tomography, magnetic resonance imaging (MRI) system, an ultrasound (US) system including contrast agents and color flow doppler, or the like.

As used herein, the terms “coordinate system” or “augmented realty system coordinate system” or “augmented reality system coordinates” refer to a 3D Cartesian coordinate system that uses one or more numbers to determine the position of points or other geometric elements unique to the particular augmented reality system or image acquisition system to which it pertains. For example, 3D points in the headset coordinate system can be translated, rotated, scaled, or the like, from a standard 3D Cartesian coordinate system.

As used herein, the terms "image data" or "imaging dataset" or "imaging data" refers to information related to an observation of the interior of the patient's body, which can include recorded imaging data, artificially created information, calculated tissue representations, or simulated portions of the patient's interior. For example, the "image data" or "imaging dataset" can include processed two-dimensional or three-dimensional images or models such as computed tomography (CT) images, magnetic resonance imaging (MRI) data, ultrasound images, fluoroscopy images, positron emission tomography (PET) images, single-photon emission computed tomography (SPECT) images, or combinations thereof, e.g., represented by data formatted according to the Digital Imaging and Communications in Medicine (DICOM) standard or other relevant imaging standards.

As used herein, the terms “imaging coordinate system” or “image acquisition system coordinate system” refers to a 3D Cartesian coordinate system that uses one or more

numbers to determine the position of points or other geometric elements unique to the particular imaging system. For example, 3D points and vectors in the imaging coordinate system can be translated, rotated, scaled, or the like, to the Augmented Reality system (head mounted displays) 3D Cartesian coordinate system.

As used herein, the terms “hologram”, “holographic,” “holographic projection”, or “holographic representation” refer to a computer-generated image stereoscopically projected through the lenses of a headset. Generally, a hologram can be generated synthetically (in an augmented reality (AR)) and is not a physical entity.

As used herein, the term “physical” refers to something real. Something that is physical is not holographic (or not computer-generated).

As used herein, the term “two-dimensional” or “2D” refers to something represented in two physical dimensions.

As used herein, the term “three-dimensional” or “3D” refers to something represented in three physical dimensions. An element that is “4D” (e.g., 3D plus a time and/or motion dimension) would be encompassed by the definition of three-dimensional or 3D.

As used herein, the term “integrated” can refer to two or more things being linked or coordinated. For example, a coil-sensor can be integrated with an interventional device.

As used herein, the term “real-time” or “near-real time” or “live” refers to the actual time during which a process or event occurs. In other words, a real-time event is done live (within milliseconds so that results are available immediately as feedback). For example, a real-time event can be represented within 100 milliseconds of the event occurring.

As used herein, the term spatial “registration” refers to steps of transforming, tracking, and imaging dataset associated with virtual representation of tracked devices – including holographic guides, applicators, and ultrasound image stream – and additional body image data for mutual alignment and correspondence of said virtual devices and image data in the head mounted displays coordinate system enabling a stereoscopic holographic projection display of images and information relative to a body of a physical patient during a procedure, for example, as further described in U.S. Patent No. 10,869,727 to Yanof et al., and also applicant’s co-owned U.S. Patent Application No. 11,701,183 to Martin III et al., the entire disclosures of which are incorporated herein by reference.

The present technology improves medical registration systems by reducing the number of required registration markers from three separate devices to a single unified device that integrates electromagnetic and optical tracking capabilities, thereby decreasing component costs, simplifying workflow, and reducing electromagnetic sensor port requirements. The technology enables flexible registration approaches that support both fiducial-based and non-fiducial anatomical registration methods, allowing operators to select appropriate techniques based on available imaging data and procedural requirements. Registration accuracy is maintained through sophisticated error handling protocols that validate geometric relationships and detect misalignment, while workflow efficiency is enhanced through automated segmentation of anatomical structures including skin and bone, enabling rapid registration without requiring pre-operative segmentation services. The system supports transitions between multiple imaging modalities including multi-detector computed tomography, cone beam computed tomography, magnetic resonance imaging, ultrasound, and optical imaging while accommodating field-of-view constraints through optimized device geometry, and enables single-step adjustment methods that allow operators to refine registration during procedures using voice commands while maintaining focus on primary procedural objectives.

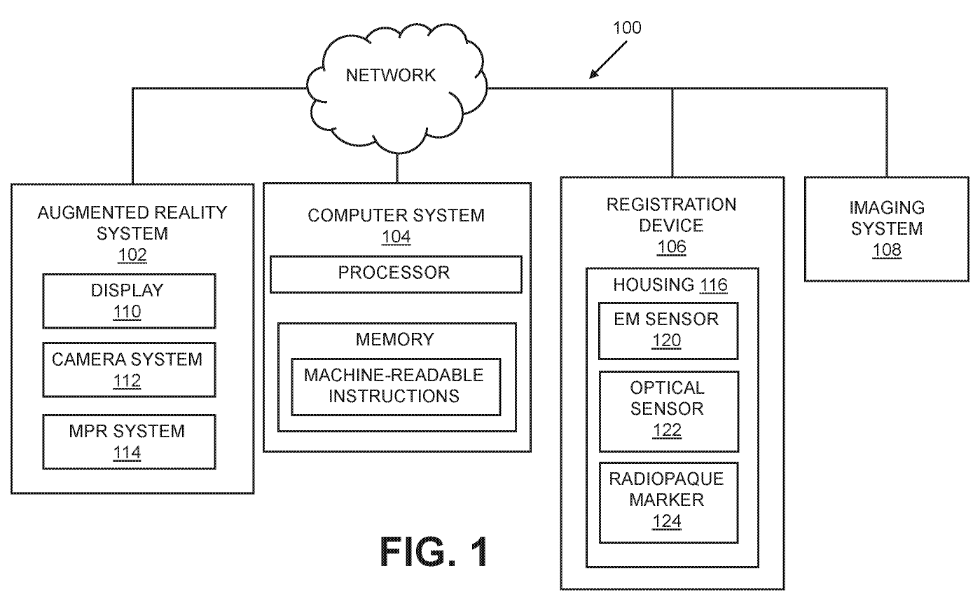

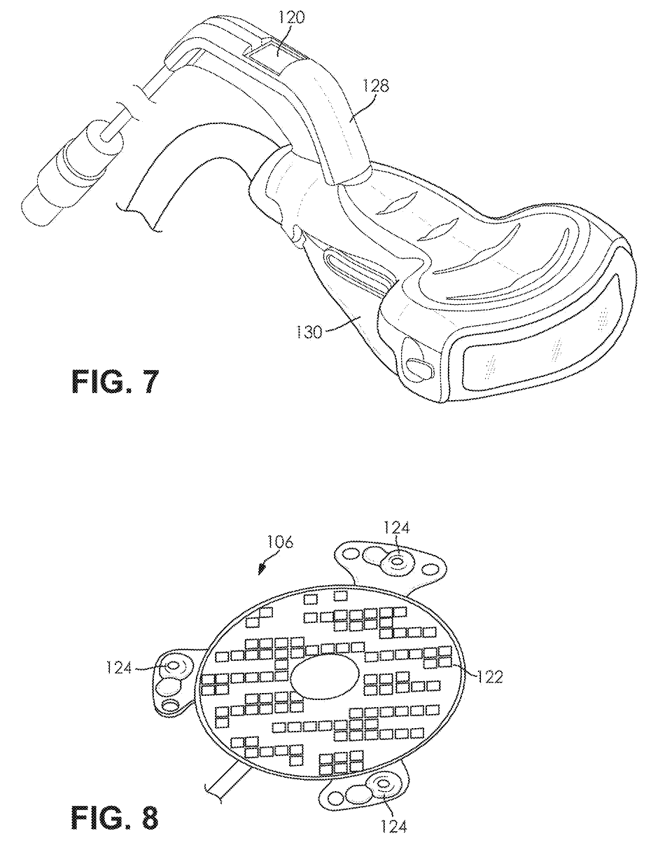

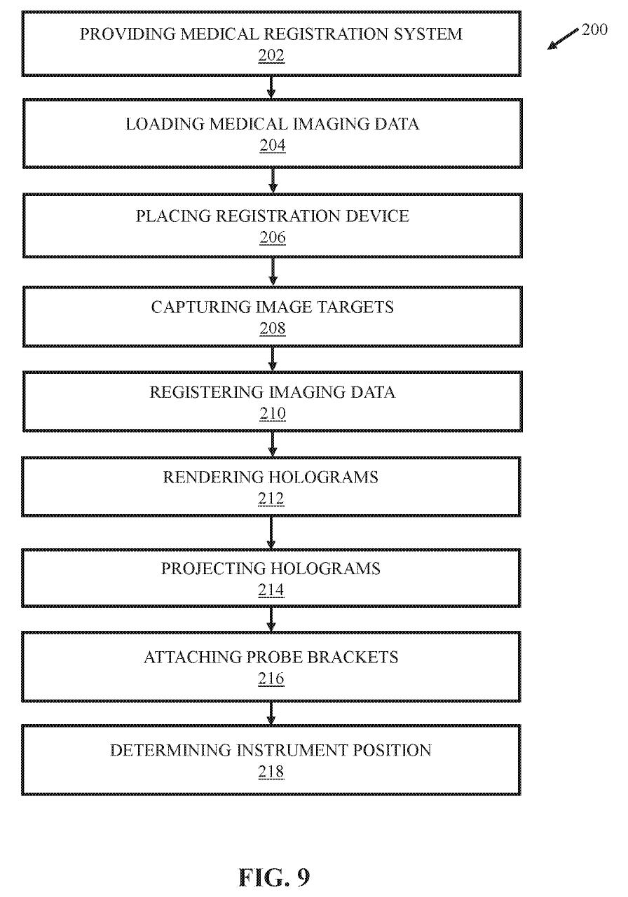

With reference to FIGS. 1-9, a medical registration system 100 for augmented reality procedural guidance is shown. The system 100 can enable operators to visualize internal anatomical structures during medical procedures by projecting holographic representations in spatial alignment with a patient's physical body. The system 100 can integrate pre-operative medical imaging data with real-time tracking of surgical instruments and ultrasound probes, providing enhanced visualization capabilities that support percutaneous interventions, surgical procedures, and diagnostic examinations. The system 100 can include an augmented reality system 102, a computer system 104, a registration device 106, and an imaging system 108, with these components functioning cooperatively to establish and maintain accurate spatial correspondence between multiple coordinate systems. The augmented reality system 102 can provide stereoscopic visualization of holographic content overlaid on the patient and surrounding physical space of the medical procedure. The computer system 104 can process medical imaging data, compute registration transformations, and coordinate data flow between system components. The registration device 106 can establish known geometric relationships that enable calculation of registration transformations. The imaging system 108 can acquire imaging datasets including tomographic data, ultrasound data, magnetic resonance imaging data, optical imaging data, or other medical imaging modalities containing anatomical information.

The augmented reality system 102 can include a display 110 configured to present holographic content or augmented reality visualizations to an operator. In certain embodiments, the display 110 can include a head-mounted display device configured to present stereoscopic holographic content to an operator while maintaining visibility of the physical environment through optical see-through visualization. Head-mounted display embodiments can be implemented using devices such as Microsoft HoloLens, Microsoft HoloLens 2, Magic Leap, or other mixed reality head-mounted displays. In alternative embodiments, the display 110 can include a non-head-mounted display device such as a computer monitor, tablet computer, smartphone, or other electronic display configured to present augmented reality visualizations through video see-through methods or as separate display windows adjacent to physical environment views. Head-mounted display embodiments can enable operators to view holographic representations superimposed on physical anatomy without requiring attention shifts to separate display monitors, while non-head-mounted display embodiments can provide augmented reality guidance through displays positioned within the field of view of the operator during procedures.

The augmented reality system 102 can include integrated camera systems 112 configured to capture images of the physical environment for multiple purposes including optical tracking, spatial mapping, and hand gesture recognition. In certain embodiments, the camera system 112 can include visible light cameras, infrared cameras, and depth sensors that collectively enable comprehensive environmental sensing. The cameras can be positioned on the head-mounted display device 110 to provide overlapping fields of view covering the region in front of the operator, typically spanning horizontal and vertical angular ranges sufficient to encompass the procedural workspace. The camera can also configured to capture images of the physical environment for optical tracking of the registration device 106. The cameras can detect and recognize optical markers 122 positioned on the registration device 106, as described in greater detail herein. The augmented reality system 102 can calculate the position and orientation of recognized optical markers in the head-mounted display coordinate system based on the captured images.

The augmented reality system 102 can include user interface capabilities enabling operator interaction through various input modalities. Voice command recognition can enable hands-free control, allowing operators to invoke functions, adjust visualization parameters, and control registration without removing sterile gloves or interrupting procedural workflow. Gesture recognition can detect hand movements, enabling operators to manipulate holographic content through pointing and manipulation gestures. Gaze tracking can detect operator viewing direction, enabling gaze-based selection of interface elements. The augmented reality system 102 can implement spatial mapping functionality to represent physical surfaces in the environment, enabling computation of occlusion relationships where holographic content positioned behind physical surfaces can be appropriately hidden or rendered with reduced visibility.

The augmented reality system 102 can operate in a float mode where holographic representations of segmented anatomical structures are displayed floating above the patient in approximate alignment based on estimated positioning prior to executing registration. Float mode can enable operators to visualize the registration device 106 placement and verify that the intended registration configuration is achievable before committing to registration execution. In float mode, the computer system 104 can estimate an approximate registration transformation based on assumptions about patient positioning on the table and typical anatomical orientations. The augmented reality system 102 can project holographic structures hovering above the patient's body with reduced opacity or distinctive visual styling to indicate that accurate registration has not yet been achieved. The operator can view the float mode holographic projections and assess the planned registration device positioning. The holographic skin surface can indicate where the registration device 106 should be placed to achieve optimal registration. The operator can physically position the registration device 106 while observing the holographic guidance, adjusting placement to align device feet with the displayed fiducial locations or anatomical landmarks. Float mode can also enable pre-registration error detection by identifying potential mismatches between the physical patient anatomy and the loaded imaging dataset, which can be acquired prior to the procedure and preloaded into the augmented reality system 102 based on patient data, or acquired contemporaneously during the procedure.

Following successful registration execution, the augmented reality system 102 can transition to a flashlight mode where holographic representations are displayed in precise spatial alignment with the physical anatomy of the patient. In flashlight mode, the computed registration transformations enable accurate projection of internal anatomical structures directly onto and within the patient's body, allowing the operator to visualize subsurface anatomy as if looking through the patient's skin. The holographic representations can be rendered at full opacity with accurate depth relationships, creating the visual effect of an ultrasound flashlight that reveals hidden anatomical structures at their true physical locations within the patient. Flashlight mode can enable real-time surgical guidance by displaying vasculature, organs, tumors, and other critical structures in correct anatomical position as the operator moves around the patient or manipulates surgical instruments. The augmented reality system 102 can maintain flashlight mode alignment throughout the procedure by continuously tracking the registration device 106 and updating the holographic projections based on any detected patient movement or registration drift.

Tracked instruments including interventional needles, ablation devices, biopsy devices, and surgical tools equipped with electromagnetic sensors can be visualized as holographic representations positioned in correct spatial alignment with the holographic anatomical structures. As instruments are advanced through the patient body, the holographic instrument representations can move correspondingly, providing the operator with visualization of the instrument position relative to internal anatomy, vascular structures, and procedural targets. The system can provide depth perception cues including stereoscopic rendering, motion parallax as the operator moves their head, occlusion relationships where instruments passing in front of anatomical structures are rendered in front, and distance indicators showing numeric measurements of separation between instrument tips and targets.

The augmented reality system 102 can include a holographic light ray (HLR) guidance system configured to enable visualization of points on both three-dimensional image projections and two-dimensional image projections during procedures. The HLR guidance system can project line segments between corresponding points on different image projections to facilitate navigation and guidance relative to anatomical targets, assisting with procedural planning and execution.

The augmented reality system 102 can include a multi-planar reconstruction (MPR) tracking system 114 configured to enable navigation through tracked spatial reconstruction of medical imaging data. The MPR tracking system 114 can generate and display MPR images in orientations corresponding to physical anatomical planes including axial, sagittal, and coronal views. These planes can be mutually perpendicular MPR holograms that intersect at an adjustable point, which can be interactively modified by the operator to update the images. Tomographic images can undergo multiplanar reformatting to be co-planar with ultrasound imaging, enabling fusion of co-planar MPR and ultrasound as a dual image modality. The ultrasound flashlight image can be co-registered with tomographic imaging data through the registration transformations computed by the computer system 104. The fused MPR and ultrasound flashlight can be co-projected with tracked interventional instruments as holographic light ray guidance relative to their physical counterparts on the patient body. This integration can enable real-time correlation between pre-operative tomographic imaging, intra-operative ultrasound imaging, and physical instrument positions, supporting accurate procedural guidance and target localization.

The computer system 104 can include at least one processor and at least one memory on which tangible, non-transitory, machine-readable instructions are stored. The processor can perform functions associated with the operation of the registration system 100 and can include one or more of general-purpose computers, special purpose computers, microprocessors, digital signal processors (DSPs), field-programmable gate arrays (FPGAs), application-specific integrated circuits (ASICs), and processors based on multi-core processor architecture. The memory can be one or more memories of any type suitable to the local application environment, and can be implemented using any suitable volatile or nonvolatile data storage technology such as semiconductor-based memory devices, magnetic memory devices and systems, optical memory devices and systems, fixed memory, and removable memory.

The memory can include random access memory (RAM), read only memory (ROM), hard disk drives (HDD), solid-state drives (SSD), or any other type of non-transitory machine or computer readable media. The instructions stored in the memory can include program instructions or computer program code that, when executed by the central processing unit (CPU) and graphics processing unit (GPU) or holographic processing unit (HPU), enable the system to perform tasks including registration processing, image segmentation, holographic rendering, and real-time tracking data integration. The computer system 104 can process registration between the electromagnetic tracking space, the head-mounted display space, and the imaging space through coordinate transformations.

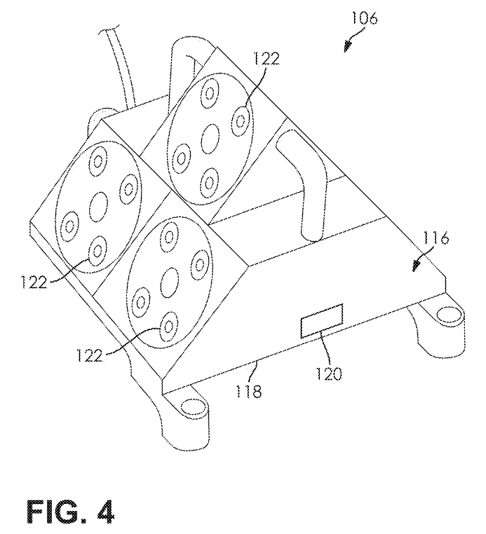

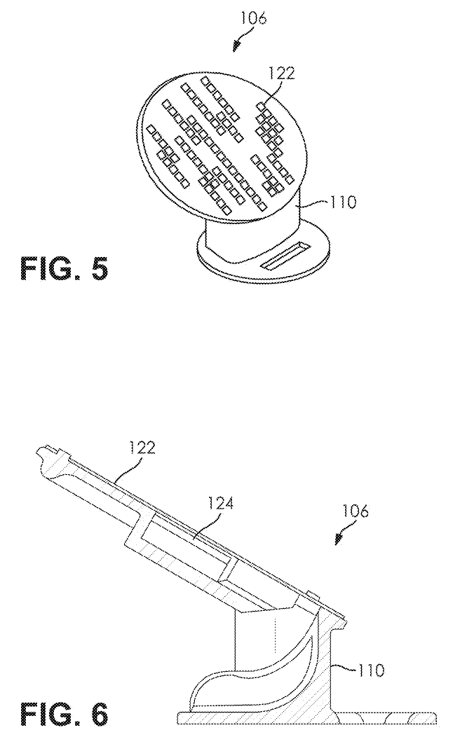

The registration device 106 can be implemented in multiple configurations to support various registration workflows and procedural requirements. In certain embodiments, the registration device 106 can be a single registration device 106 that integrates multiple optical tracking targets with electromagnetic tracking capabilities, for example, as shown in FIG. 4. In alternative embodiments, the registration device 106 can include multiple discrete units positioned independently on the patient anatomy according to predetermined geometric patterns, for example as shown in FIGS. 5-6.

The system 100 can include an electromagnetic field generator positioned within the procedural workspace to create a magnetic field that enables the electromagnetic sensors 120 to determine their position and orientation. The electromagnetic field generator can be implemented as a tabletop generator mounted beneath the patient table, a planar generator positioned adjacent to the patient, or other electromagnetic field generator configurations compatible with the procedural environment. Regardless of configuration, the registration device 106 can establish known geometric relationships between electromagnetic tracking coordinates and optical tracking coordinates. Electromagnetic tracking coordinates refer to the precise three-dimensional position (x, y, z) and orientation (pitch, yaw, roll) data of the electromagnetic sensor 120 determined by analyzing electromagnetic field strengths generated by the electromagnetic field generator. These electromagnetic tracking coordinates, combined with optical tracking coordinates from the optical markers 122, enable computation of registration transformations between the electromagnetic tracking coordinate system, the head-mounted display coordinate system, and the tomographic imaging coordinate system.

The registration device 106 can include a housing 116 configured to support and protect the tracking components. The housing 116 can have a planar or contoured surface configured to conform to patient anatomy. The housing 116 can include a patient contact surface 118 configured for placement on skin or anatomical landmarks. The contact surface 118 can include adhesive regions for securing the device to the patient, or mounting interfaces for attachment to adjustable positioning systems. The patient contact surface 118 can include one or more foot structures extending from the platform body for contact with the patient skin at anatomically significant locations. The foot structures can have varying geometric configurations, shapes, orientations, and colors optimized for anatomical contours and optical recognition. The contact surface 118 can be positioned at locations that correspond to fiducial markers visible in pre-operative imaging, or at anatomically identifiable landmarks such as the xiphoid process, sternum, costal margins, or other palpable anatomical features, as non-limiting examples.

The registration device 106 can include various combinations of one or more electromagnetic sensors 120, one or more optical markers 122, and one or more radiopaque fiducial markers 124. The electromagnetic sensors 120 can be positioned within or on the housing 116 and can generate position and orientation data in an electromagnetic tracking coordinate system. The electromagnetic sensors 120 can be 5-degree-of-freedom (5DOF) sensors that measure position (three translational coordinates) and partial orientation (two rotational coordinates), or 6-degree-of-freedom (6DOF) sensors that measure both position (three translational coordinates) and full orientation (three rotational coordinates). The electromagnetic sensors 120 can be compatible with electromagnetic field generators such as the Aurora electromagnetic tracking system from Northern Digital Inc. The optical markers 122 can be positioned on the housing and can enable optical tracking in the head-mounted display coordinate system. The optical markers 122 can display visual patterns including Vuforia image targets, ArUco markers, AprilTag patterns, Advanced Model Target (AMT) features, or other optically detectable codes. The radiopaque markers 124 can be integrated within or on the housing 116 and can enable detection in tomographic imaging coordinate systems including computed tomography (CT), cone beam computed tomography (CBCT), and magnetic resonance imaging (MRI). The registration device 106 can include detachable sensor assemblies that enable separation of reusable tracking components from disposable patient-contact elements. The electromagnetic sensor 120 can be housed in a reusable module that attaches to a disposable registration device 106, or a disposable optical tracking component can snap onto a reusable electromagnetic sensor assembly.

The housing 116 can have a rigid structure that maintains fixed spatial relationships between components. The one or more electromagnetic sensors 120, one or more optical markers 122, and one or more radiopaque markers 124 can be positioned at known locations within or on the housing 116. Because the housing is rigid 116, the geometric offsets between these components remain constant. Geometric offset data can be measured during manufacturing and embedded in the registration device 106, for example, through machine-readable codes such as QR codes, RFID tags, or NFC chips as non-limiting examples. When the registration device 106 is prepared for use, the computer system 104 can automatically read the embedded calibration data and load the geometric offset values for that specific device, reducing manual device selection steps and reducing potential for operator error. Because the spatial relationships between the electromagnetic sensors 120, optical markers 122, and radiopaque markers 124 are fixed and known to the system 100, the computer system 104 can calculate transformations that align the electromagnetic tracking coordinate system, the head-mounted display coordinate system, and the tomographic imaging coordinate system. These known geometric relationships enable the computer system 104 to calculate positions across coordinate systems based on tracking data from any of the integrated sensors or markers.

In certain embodiments, the registration device 106 can be provided as a set of multiple discrete registration devices. Each unit in the set can include a housing 116 containing an electromagnetic sensor 120 and an optical marker 122, and can be independently positionable on the patient anatomy. In one particular embodiment, the registration device 106 can be provided as a set of three units. In operation, the three units can be positioned according to a predetermined geometric pattern, such as a triangle configuration, with each unit placed at a location corresponding to a fiducial marker visible in pre-operative imaging or at anatomically identifiable landmarks. A skilled artisan can select a suitable number and arrangement of registration devices within the scope of the present disclosure.

In other embodiments, the registration device 106 can include a unified body structure that integrates multiple optical tracking targets 122 with one or more electromagnetic sensors 120 in a registration device. The registration device 106 can be shaped as a triangle with a midline axis oriented to align with anatomical midline structures such as the sternum or spine according to one non-limiting embodiment. The registration device 106 can integrate three or more optical tracking targets 122 at predetermined locations, for example at vertices and central regions of the housing 116. The registration device 106 can include labeling features that provide anatomical orientation information, such as "LEFT" and "RIGHT" labels corresponding with patient laterality, a "MIDLINE" delineation aligned with the longitudinal axis, and "ANTERIOR" labeling indicating the superior surface.

Triangle configurations can vary based on imaging modality and field-of-view constraints. Scalene triangle templates having unequal side lengths can be optimized for standard multi-detector computed tomography (MDCT) field-of-view requirements. Right-angle triangle templates can be optimized for cone beam computed tomography (CBCT) applications where field-of-view constraints differ from MDCT systems. Isosceles triangle configurations can be particularly advantageous when used with unified registration device bodies that integrate multiple optical markers 122 on a single platform, as the symmetric geometry along the midline can facilitate alignment with anatomical midline structures such as the sternum or spine.

The system 100 can include one or more probe brackets 128 configured to integrate electromagnetic or optical tracking capabilities with a medical instrument 130 or device that do not include integral tracking sensors. The probe bracket 128 can be configured for attachment to ultrasound probes, surgical instruments, ablation devices, biopsy needles, or other medical devices as selected by a skilled artisan within the scope of the present disclosure. The probe bracket 128 can include attachment features configured to securely engage the instrument without interfering with clinical functionality. The attachment features can include snap-fit mechanisms, clamp structures, or adhesive interfaces that enable repeatable attachment and removal.

The probe bracket 128 can attach to the medical instrument 130 and include tracking components such as an optical marker 122 and/or an electromagnetic sensor 120 positioned at known locations within or on the bracket body on the probe bracket 128. By providing these tracking components, the probe bracket 128 enables the system 100 to determine the position and orientation of the attached instrument 130 within the electromagnetic tracking coordinate system or the head-mounted display coordinate system. When the probe bracket 128 is attached to the medical device 130, the system 100 can determine the location of the tracking components relative to the device itself based on the known geometric relationships. The computer system 104 can calculate the position and orientation of the medical device based on tracking data from the sensors 120 and/or markers 122 and the known geometric offsets between the tracking components and the device.

Because the electromagnetic sensors 120 and optical markers 122 are positioned at known locations within the rigid housing 116 of the registration device 106, the computer system 104 can measure the same physical points in both electromagnetic tracking coordinates and head-mounted display (HMD) coordinates, enabling computation of rigid transforms consisting of three-dimensional rotation and translation. The computer system 104 can calculate two primary transforms to align the coordinate systems. An electromagnetic-to-head-mounted- display transform relates positions measured in the electromagnetic tracking coordinate system to positions in the head-mounted display coordinate system. A tomographic-to-head-mounted-display transform relates positions in tomographic imaging coordinates to positions in the head-mounted display coordinate system. The computer system 104 can maintain both transforms simultaneously, enabling real-time fusion of pre-operative tomographic imaging data with intra-operative ultrasound imaging and tracked instrument positions.

To compute the electromagnetic-to-head-mounted-display transform, the computer system 104 can measure the registration device 106 in both electromagnetic tracking coordinates (via the electromagnetic sensors 120) and head-mounted display coordinates (via the optical markers 122). The corresponding point sets enable computation of the transform using point-based rigid registration algorithms, including singular value decomposition (SVD) methods that compute optimal rotation and translation parameters minimizing residual error between corresponding point sets as a non-limiting example.

To compute the tomographic-to-head-mounted-display transform, the computer system 104 can identify point locations on the registration device 106 that are visible in both tomographic imaging (e.g., via radiopaque markers 124 or anatomical landmarks) and electromagnetic tracking coordinates. By applying the electromagnetic-to-head-mounted-display transform to the electromagnetic coordinates, the computer system 104 can determine the corresponding head-mounted display coordinates. The point correspondences between tomographic coordinates and head-mounted display coordinates enable computation of the tomographic-to-head-mounted-display transform, allowing stereoscopic projection of tomographic imaging to align with the physical body.

For registration devices with a single electromagnetic sensor 120 and multiple optical markers 122, the computer system 104 can implement geometric offset transformations to calculate optical marker positions based on stored geometric offset vectors defining the spatial relationships between the sensor and each optical marker. The computer system 104 can apply the sensor rotation data to transform the offset vectors into the electromagnetic tracking coordinate system before adding them to the sensor position to determine the global position of each optical marker. Similarly, for probe brackets 128, the computer system 104 can calculate tracked ultrasound imaging plane positions relative to electromagnetic sensor locations based on stored geometric offsets. The computer system 104 can maintain calibration data for each registration device configuration, storing the specific geometric offset values that relate sensor positions to optical marker centroids and to anatomical reference points. The calibration data can be embedded in machine-readable codes on the registration device 106 or probe bracket 128 and automatically loaded by the computer system 104 during device preparation.

The computer system 104 can load and process medical imaging data from various imaging modalities including computed tomography (CT), magnetic resonance imaging (MRI), cone beam computed tomography (CBCT), positron emission tomography (PET), and ultrasound systems. The imaging data can be formatted according to the Digital Imaging and Communications in Medicine (DICOM) standard or other imaging standards.

The computer system 104 can perform automatic segmentation of anatomical structures including skin surfaces, bone surfaces, organs, vascular structures, tumors, and fiducial markers. Segmentation algorithms can include threshold-based methods, region growing algorithms, edge detection methods, machine learning classifiers, deep learning neural networks, and combinations thereof, as non-limiting examples. The computer system 104 can prioritize automatic segmentation in a predetermined order, for example, first skin and bone structures, then fiducial markers, followed healthy organs, and then pathological tissues or tumors.

For fiducial marker detection, the computer system 104 can process imaging data acquired by the imaging system 108 to identify radiopaque markers 124. The computer system 104 can utilize threshold-based segmentation to identify high-intensity regions in the imaging data corresponding to radiopaque markers. The computer system 104 can perform connected component analysis to identify discrete marker instances and calculate the centroid or geometric center of each detected marker as the fiducial position in imaging coordinates. The computer system 104 can verify that the expected number of fiducial markers has been detected and that the geometric configuration matches the template pattern.

For anatomical landmark registration workflows, the computer system 104 can perform automatic segmentation of skin and bone surfaces from tomographic imaging. Skin segmentation can utilize threshold-based methods that identify the boundary between air and soft tissue, followed by morphological operations including dilation and erosion to clean the masks and smooth the surface. Bone segmentation can utilize threshold-based methods that identify voxels with intensity values exceeding bone thresholds, followed by connectivity analysis to separate distinct bone structures. Segmented surfaces can be represented as polygon meshes, point clouds, or implicit surface representations suitable for rendering and registration algorithms. The computer system 104 can enable interactive segmentation refinement, allowing operators to manually adjust automatically generated segmentation results.

The computer system 104 can implement error checking processes to ensure registration accuracy. Prior to registration, the computer system 104 can implement triangle compatibility checks that compare geometric relationships between optical markers captured by the optical tracking system and the expected relationships based on electromagnetic sensor-derived positions. Following registration, the computer system 104 can calculate Fiducial Registration Error (FRE) as the RMS value of distances between corresponding points after applying the registration transformation. In certain embodiments, FRE thresholds of approximately 2mm can be used, consistent with clinical requirements for percutaneous procedures. The computer system 104 can implement continuous validation checks that monitor registration stability throughout procedures, distinguishing between registration device 106 movement (which invalidates registration) and patient gross motion. When tracking quality degradation is detected, the computer system 104 can identify potential causes and alert the operator.

The computer system 104 can support interactive registration adjustment procedures that enable operators to refine registration accuracy during procedures. Voice command controls can enable hands-free adjustment of registration parameters without requiring the operator to remove sterile gloves or interrupt procedural workflow. Voice commands can include instructions such as "adjust registration," "move anatomy left," "move anatomy anterior," or "rotate anatomy clockwise," which can invoke adjustment interfaces. The registration adjustment interface can enable the operator to apply translational and rotational offsets to the holographic projections, with holographic projections updating in real-time. The system can record all registration adjustments applied during procedures, maintaining a log of adjustment timing, magnitude, and direction. For procedures utilizing tracked ultrasound imaging, the system can support ultrasound-based registration refinement where the operator matches ultrasound image features with corresponding features in tomographic imaging to refine registration based on direct image comparison.

The computer system 104 can implement motion compensation algorithms that distinguish between different types of patient movement, including respiratory motion, cardiac motion, gross patient repositioning, and registration device displacement. For procedures involving respiratory motion, the computer system 104 can detect periodic motion patterns from the electromagnetic sensor data and apply real-time transform updates that maintain holographic alignment throughout the respiratory cycle. Machine learning models can be trained on datasets correlating external registration device positions with internal anatomical feature positions, enabling prediction of organ and tissue locations based on skin surface markers even when respiratory motion or patient movement occurs.

The computer system 104 can record three-dimensional images for documentation and reimbursement purposes, capturing spatial information about anatomical features, instrument positions, and procedural events. The recording capabilities can include volumetric imaging datasets, tracking data for users and devices throughout procedures, and video for procedural playback. The recorded data can be timestamped and synchronized across multiple data streams including imaging, tracking, and user interaction events. The computer system 104 can maintain a comprehensive procedural log that documents registration events, tracking data quality metrics, imaging acquisitions, instrument usage, and operator actions. Tracking data recorded throughout procedures can be analyzed post-procedurally to assess procedural efficiency metrics including time to target engagement, number of instrument repositioning attempts, and total procedure duration. Registration quality metrics including FRE and TRE can be recorded along with registration execution timestamps for post-procedural analysis and quality assurance.

The computer system 104 can maintain communication with the augmented reality system 102 and registration device 106 through various network configurations including local area networks (LANs), wide area networks (WANs), and wireless local area networks (WLANs). The computer system 104 can communicate with electromagnetic tracking systems through dedicated tracking system interfaces, receiving real-time 6-degree-of-freedom position and orientation data for multiple sensors simultaneously. The computer system 104 can communicate with imaging systems 108 through DICOM networking protocols to receive imaging datasets from imaging modalities or picture archiving and communication systems (PACS).

The imaging system 108 can be configured to generate imaging datasets of a patient anatomical features. The imaging system 108 can include various imaging modalities such as computed tomography (CT) systems, magnetic resonance imaging (MRI) systems, cone beam computed tomography (CBCT) systems, positron emission tomography (PET), ultrasound systems, fluoroscopy systems, and combinations thereof. The imaging system 108 can be utilized to generate imaging datasets before a procedure, during a procedure, after a procedure, or in combinations thereof. Pre-operative imaging can provide anatomical information for procedural planning, target identification, and registration preparation. Intra-operative imaging can enable real-time verification of instrument positioning and confirmation of procedural progress. Post-operative imaging can document procedural results and verify target treatment.

The system 100 can integrate imaging datasets from multiple imaging modalities to leverage complementary information. Pre-operative CT imaging can provide anatomical detail with bone visualization. Pre-operative MRI imaging can provide soft tissue contrast, enabling visualization of tumors, vascular structures, and organs. PET imaging can provide metabolic information characterizing tumor activity or other physiological processes. The computer system 104 can perform multi-modality image registration to align imaging datasets acquired with different modalities. Following multi-modality registration, the computer system 104 can enable fusion visualization where information from multiple imaging modalities is displayed simultaneously. For example, PET metabolic data can be overlaid on CT anatomical structures, or MRI tumor segmentations can be projected onto CT imaging datasets. The registration device 106 can enable alignment of all pre-registered imaging modalities with the patient physical anatomy through a single registration procedure. Once the registration transformation between tomographic imaging and head-mounted display coordinates is computed based on CT imaging containing visible fiducial markers or registration device features, the computer system 104 can apply the same transformation to co-registered MRI and PET datasets. This enables holographic projection of multi-modality imaging data with consistent spatial alignment to the patient anatomy.

Different imaging modalities can provide varying field-of-view coverage that affects registration device selection and positioning. Multi-detector computed tomography (MDCT) systems can provide relatively large field-of-view coverage, enabling imaging of registration devices with widely spaced fiducial markers. Cone beam computed tomography (CBCT) systems, can employed for intra-operative imaging, can have smaller fields of view compared to MDCT systems. The registration device 106 can be selected based on the field-of-view constraints of the imaging modality, with more compact device configurations suitable for imaging systems with constrained fields of view. In certain embodiments utilizing unified registration device bodies with integrated optical markers 122, the device footprint can be reduced compared to three separate Advanced Combo Markers while maintaining adequate optical marker separation for accurate registration calculations. The integration of multiple optical markers 122 on a single rigid housing 116 can enable registration within field-of-view limitations while preserving registration accuracy.

The imaging datasets can be formatted according to the Digital Imaging and Communications in Medicine (DICOM) standard or other imaging standards. The computer system 104 can be in communication with the imaging system 108 via networks including local area networks (LANs), hospital information systems (HIS), radiology information systems (RIS), and picture archiving and communication systems (PACS).

In one embodiment, a system 100 for augmented reality visualization during medical procedures on a patient 110, comprises: an augmented reality display system 102 configured to display representations of internal anatomical structures of the patient 110 overlaid in spatial alignment with physical anatomy of the patient 110; a registration device 106 configured to be positioned on the patient 110, the registration device 106 including a tracking component (e.g., electromagnetic sensor 120, optical marker 122) that generates tracking data indicating position and orientation of the registration device 106 relative to the patient 110; an imaging system 108 configured to acquire medical imaging data of internal anatomical structures of the patient 110; a computer system 104 configured to: receive the medical imaging data from the imaging system 108, receive the tracking data from the registration device 106, determine spatial alignment between the medical imaging data and the patient 110 based on the tracking data and a known position of the registration device 106 on the patient 110, compute registration transformations between an imaging coordinate system and a physical coordinate system of the patient 110, and transform the medical imaging data using the registration transformations to enable the augmented reality display system 102 to display the holographic representations in correct spatial alignment with the physical anatomy of the patient 110 during medical procedures.

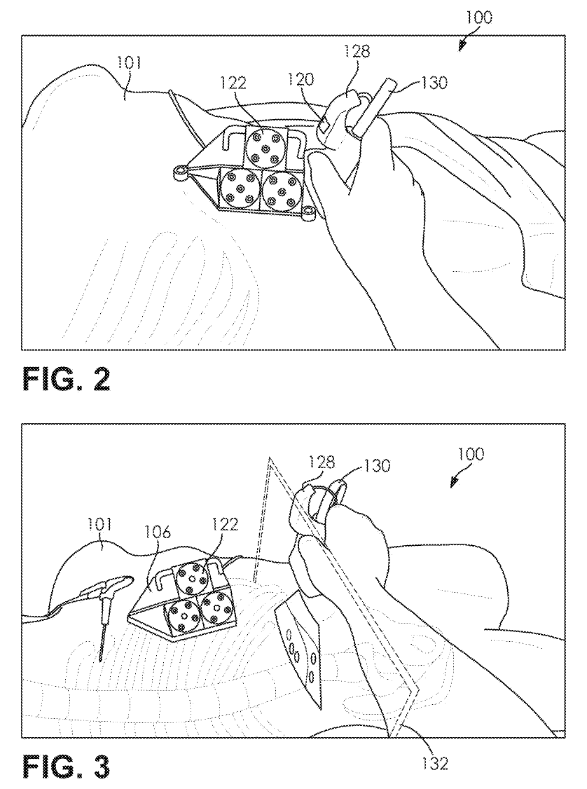

With reference to FIGS. 2-3 and 9, a method 200 for using the medical registration system 100 is shown. The method 200 provides a unified workflow with adaptive steps that accommodate different imaging data availability and procedural requirements. The method 200 can begin with step 202 of providing the medical registration system 100 including the augmented reality system 102, the computer system 104, the registration device 106, and the imaging system 108.

In step 204, the computer system 104 can load medical imaging data from the imaging system 108 and perform automatic segmentation of anatomical structures. The segmentation approach can adapt based on available imaging data. When radio-opaque fiducial markers 124 are present in the pre-operative imaging, the computer system 104 can identify and segment the radio-opaque fiducial markers 124 to enable correlation between physical marker positions and their corresponding locations in tomographic imaging data. When radio-opaque fiducial markers 124 are not present or are insufficient, the computer system 104 can perform automatic segmentation of skin and bone surfaces from tomographic imaging data to provide reference surfaces that can be correlated with corresponding physical anatomical surfaces on the patient during registration.

In step 206, the registration device 106 can be physically placed on the patient 101. The placement approach can adapt to correspond with the segmentation performed in step 204. When fiducial markers are present in the imaging, the registration device 106 can be positioned according to the external fiducial marker locations. When fiducial markers are not present or are insufficient, the registration device 106 can be positioned using identifiable anatomical landmarks such as the xiphoid process, umbilicus, sternum, costal margins, or anterior superior iliac crests that correspond to the automatically segmented anatomical surfaces. Hybrid approaches can combine available fiducial marker information with anatomical landmark positioning when one or two fiducial markers are present.

In step 208, the system can capture the optical markers 122 on the registration device 106 and determine the position of the registration device 106 relative to the patient based on both electromagnetic tracking data from the electromagnetic sensor 120 and optical tracking data from the optical markers 122. For registration devices with a single electromagnetic sensor 120 and multiple optical markers 122, the computer system 104 can calculate the positions of the optical markers 122 based on the electromagnetic sensor position combined with stored geometric offset vectors that represent the fixed spatial relationships between the electromagnetic sensor 120 and each optical marker 122. The computer system 104 can perform triangle compatibility checks by comparing the triangle formed by the three optical marker positions as measured by the optical tracking system with the triangle formed by the three optical marker positions as calculated from the electromagnetic sensor and geometric offsets. If the triangle compatibility error exceeds predetermined thresholds, the system can alert the operator and prompt re-capture of optical markers or verification of registration device positioning.

In step 210, upon successful optical marker capture and validation of triangle compatibility, the computer system 104 can register the imaging data to the patient based on the determined position of the registration device 106. The registration process can establish correspondence between the electromagnetic tracking coordinate system, the head-mounted display coordinate system, and the tomographic imaging coordinate system. The correspondence can be established using either the fiducial marker positions identified in step 204 and the corresponding device placement in step 206, or using the anatomical surface features identified in step 204 and the corresponding device placement in step 206. The computer system 104 can compute registration transformations that enable conversion of coordinates between these coordinate systems, allowing holographic projections to be accurately positioned relative to patient anatomy. Following registration completion, the computer system 104 can calculate registration error metrics including Fiducial Registration Error (FRE), Registered Triangle Error (RTE), and Target Registration Error (TRE). If error metrics exceed acceptable thresholds, the computer system 104 can alert the operator and prompt corrective actions.

In step 212, the augmented reality system 102 can render one or more holograms 132 based on the registered imaging dataset. The computer system 104 can apply the computed registration transformations to convert imaging data and tracking data into the head-mounted display coordinate system for rendering. The augmented reality system 102 can generate holographic representations including segmented anatomical structures, holographic light ray (HLR) guidance showing directional guidance for instrument insertion, multi-planar reconstruction (MPR) displays showing anatomical plane orientations, and ultrasound flashlight images co-registered with tomographic imaging.

In step 214, the augmented reality system 102 can project the rendered holograms in the augmented reality environment. The projection can update dynamically in response to operator movement, patient movement, and tracked instrument movement. Tracked instruments equipped with electromagnetic sensors can be visualized as holographic representations positioned in correct spatial alignment with the holographic anatomical structures, providing operators with visualization of instrument positions relative to internal anatomy, vascular structures, and procedural targets.

In step 216, one or more probe brackets 128 can be attached to medical instruments 130 that do not include integral tracking sensors. The probe bracket 128 can attach to ultrasound probes, surgical instruments, ablation devices, biopsy needles, or other medical devices. The probe bracket 128 can include tracking components such as an optical marker 122 or an electromagnetic sensor 120 positioned at known locations within the bracket body. The attachment features can include snap-fit mechanisms, clamp structures, or adhesive interfaces that enable secure engagement with the instrument without interfering with clinical functionality.

In step 218, the computer system 104 can determine the position and orientation of the attached medical instrument 130 based on tracking data from the sensors 120 or markers 122 on the probe bracket 128 and the known geometric offsets between the tracking components and the device. When the probe bracket 128 is attached to the medical device 130, the system 100 can calculate the position and orientation of the medical device based on the tracking data and the known geometric relationships stored in the calibration data for that specific probe bracket configuration. The augmented reality system 102 can render and project holographic representations of the tracked medical instruments 130 in real-time spatial alignment with the holographic anatomical structures and the physical patient. As instruments are advanced through the patient body, the holographic instrument representations can move correspondingly, providing the operator with visualization of the instrument position relative to internal anatomy, vascular structures, and procedural targets.

Following registration execution and holographic projection, the electromagnetic sensor 120 within the registration device 106 can continuously monitor the position and orientation of the registration device 106 within the magnetic field generated by the electromagnetic field generator, enabling detection of device movement that could invalidate registration. The system 100 can distinguish between registration device movement and patient gross motion through analysis of tracking data patterns. If the registration device 106 moves together with the patient due to table adjustments or patient repositioning, the registration may remain valid if the spatial relationship between the registration device and the patient anatomy is preserved. For procedures involving respiratory motion, the electromagnetic sensor 120 within the registration device 106 can detect periodic motion patterns characteristic of respiration while positioned within the magnetic field, and the computer system 104 can apply real-time transform updates that maintain holographic alignment throughout the respiratory cycle.

In one embodiment, a method 200 for medical registration, comprises: positioning 206 a registration device 106 on a patient 110, the registration device 106 including a tracking component (e.g., electromagnetic sensor 120, optical marker 122); determining a position of the registration device 106 relative to the patient 110 based on tracking data from the tracking component; registering 210 medical imaging data to the patient 110 based on the determined position of the registration device 106; determining 218 position and orientation of a medical instrument 130 based on tracking data from a tracking component in a probe bracket 128 attached to the medical instrument 130; and displaying 214, by an augmented reality system 102, augmented representations based on the registered medical imaging data and the position and orientation of the medical instrument 130.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A system for augmented reality visualization during medical procedures on a patient, comprising:
an augmented reality display system configured to display representations of internal anatomical structures of the patient overlaid in spatial alignment with physical anatomy of the patient;
a registration device configured to be positioned on the patient, the registration device including a tracking component that generates tracking data indicating position and orientation of the registration device relative to the patient;
an imaging system configured to acquire medical imaging data of internal anatomical structures of the patient;
a computer system configured to:
receive the medical imaging data from the imaging system,
receive the tracking data from the registration device,
determine spatial alignment between the medical imaging data and the patient based on the tracking data and a known position of the registration device on the patient,
compute registration transformations between an imaging coordinate system and a physical coordinate system of the patient, and
transform the medical imaging data using the registration transformations to enable the augmented reality display system to display the holographic representations in correct spatial alignment with the physical anatomy of the patient during medical procedures.

2. The system of claim 1, wherein the tracking component of the registration device comprises an optical marker, an electromagnetic sensor, or a radiopaque marker.

3. The system of claim 2, wherein the registration device includes a combination of two or more tracking components selected from the group consisting of an optical marker, an electromagnetic sensor, and a radiopaque marker.

4. The system of claim 1, further comprising one or more probe brackets configured to attach to a medical instrument, the probe bracket including a tracking component and the computer system is configured to determine position and orientation of the medical instrument based on tracking data from the tracking components on the probe bracket.

5. The system of claim 4, wherein the tracking component on the probe bracket includes one of an optical marker or an electromagnetic sensor.

6. The system of claim 4, wherein the medical instrument comprises an ultrasound probe, a surgical instrument, an ablation device, or a biopsy needle.

7. The system of claim 4, wherein the augmented reality system is configured to render and project holographic representations of the tracked medical instruments in real-time spatial alignment with holographic anatomical structures and the physical patient.

8. The system of claim 1, wherein the computer system is configured to perform automatic segmentation of anatomical structures including at least one of skin surfaces, bone surfaces, organs, vascular structures, tumors, and fiducial markers.

9. The system of claim 8, wherein the augmented reality system is configured to render holograms of the segmented anatomical structure.

10. The system of claim 1, wherein the augmented reality system includes a head-mounted display device configured to present stereoscopic holographic content.

11. The system of claim 1, wherein the augmented reality system includes a multi-planar reconstruction tracking system configured to generate and display multi-planar reconstruction images in orientations corresponding to anatomical planes.

12. The system of claim 1, wherein the augmented reality system includes user interface capabilities enabling operator interaction through one of voice command recognition, gesture recognition, and gaze tracking.

13. The system of claim 1, wherein the computer system is configured to implement motion compensation algorithms that distinguish between respiratory motion, cardiac motion, gross patient repositioning, and registration device displacement.

14. The system of claim 1, wherein the computer system is configured to integrate imaging datasets from multiple imaging modalities and perform multi-modality image registration to align imaging datasets acquired with different modalities.

15. The system of claim 1, wherein the imaging system includes one of a computed tomography system, a magnetic resonance imaging system, a cone beam computed tomography system, or an ultrasound imaging system.

16. The system of claim 1, further comprising another imaging system, the another imaging system including one of a computed tomography system, a magnetic resonance imaging system, a cone beam computed tomography system, or an ultrasound imaging system, and wherein the another imaging system comprises a different imaging modality than the imaging system.

17. A method for medical registration, comprising:
positioning a registration device on a patient, the registration device including a tracking component;
determining a position of the registration device relative to the patient based on tracking data from the tracking component;
registering medical imaging data to the patient based on the determined position of the registration device;
determining position and orientation of a medical instrument based on tracking data from a tracking component in a probe bracket attached to the medical instrument; and
displaying, by an augmented reality system, augmented representations based on the registered medical imaging data and the position and orientation of the medical instrument.

18. The method of claim 17, wherein positioning the registration device on the patient using anatomical landmarks includes positioning the registration device at an anatomically identifiable landmark.

19. The method of claim 17, further comprising continuously monitoring position and orientation of the registration device using the tracking components, and detecting device movement.

20. The system of claim 17, wherein the tracking component of the registration device is one of an optical marker, an electromagnetic sensor, and a radiopaque marker.
